(19) United States
(12) Patent Application Publication
(10) Pub. No.: US 2016/0008147 A1
(43) Pub. Date: Jan. 14, 2016

(54) PROSTHETIC SPORT FEET

(71) Applicant: Össur hf, Reykjavik (IS)

(72) Inventor: Dana Stewart Marlin, Hafnarfjordur (IS)

(21) Appl. No.: 14/798,338

(22) Filed: Jul. 13, 2015

Related U.S. Application Data
(60) Provisional application No. 62/024,335, filed on Jul. 14, 2014.
(60) Provisional application No. 62/113,129, filed on Feb. 6, 2015.

(51) Int. Cl. A61F 2/66

(52) U.S. Cl. CPC A61F 2/66; A61F 2002/6621; A61F 2002/6642; A61F 2002/6657

(57) ABSTRACT

Various features for improving the performance of prosthetic sport feet are provided. Prosthetic sport feet according to the present disclosure can be designed to improve performance in board sport activities, for example, surfing, windsurfing, kitesurfing, wakeboarding, skateboarding, snowboarding, skiing, and the like. A prosthetic board sports foot can have a wide profile and large area for contacting the board or ground to improve stability. The board sports foot can include a traction sole to reduce slippage on the board. The board sports foot can also include an attachment member for attaching the foot to a user's residual limb or another prosthetic component. The attachment member can include a tendon designed to allow for flexibility over an initial short range of movement. The tendon can be interchangeable, and tendons having various stiffnesses can be provided.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the priority benefit of U.S. Provisional Application Nos. 62/024,335, filed Jul. 14, 2014, and 62/113,129, filed Feb. 6, 2015, the entireties of which are hereby incorporated by reference herein and should be considered part of this specification.

### BACKGROUND

1. Field

The present application relates to foot prostheses in general, and more particularly, to prosthetic sport feet designed for board sport activities.

2. Description of the Related Art

Various types of prosthetic foot devices are available as substitutes for human feet. Some prosthetic feet are designed especially for sporting activities such as running, both at the recreational and competitive levels. However, some prosthetic foot users could benefit from prosthetic feet designed for other sporting activities.

### SUMMARY

In some embodiments, a prosthetic foot includes an elongate foot member having a toe portion, a heel portion, and a substantially horizontal arch portion between the toe portion and the heel portion. The toe portion includes a toe ground contacting portion, and the heel portion includes a heel ground contacting portion. A width of the toe portion increases from the arch portion to the toe ground contacting portion, and a width of the heel portion increases from the arch portion to the heel ground contacting portion.

In some embodiments, the toe and heel portions are symmetrical in a medial-lateral direction, and the toe portion is symmetrical to the heel portion. The toe portion and heel portion can have substantially the same length as measured from a center of the arch portion. In other embodiments, the toe portion is longer than the heel portion. The toe ground contacting portion and heel ground contacting portion can have substantially the same width. The toe and heel portions also have substantially the same size and shape. In some embodiments, the toe and heel portions have openings therethrough. The prosthetic foot can further include an attachment member coupled to the arch portion. The attachment member is configured to couple the foot member to a user's residual limb or a prosthetic component.

In some embodiments, a prosthetic foot includes a foot member and an attachment member. The foot member includes a substantially horizontal arch portion, a toe portion extending downward and forward from the arch portion to a toe ground contacting portion at a toe end of the toe portion, and a heel portion extending downward and rearward from the arch portion to a heel ground contacting portion at a heel end of the heel portion. A width of the toe portion increases from the arch portion to the toe ground contacting portion, and a width of the heel portion increases from the arch portion to the heel ground contacting portion. The attachment member is coupled to the arch portion and is configured to couple the foot member to a user's residual limb or a prosthetic component. The attachment member includes a lower attachment element coupled to the arch portion, an upper attachment element including a connector configured to couple to the user's residual limb or the prosthetic component, and a resilient tendon extending between and coupled to the upper and lower attachment elements.

In some embodiments, the toe portion and heel portion are symmetrical. The toe and heel portions can have substantially the same length as measured from a center of the arch portion. In other embodiments, the toe portion is longer than the heel portion. In some embodiments, the toe ground contacting portion and heel ground contacting portion have substantially the same width. The toe and heel portions can also have substantially the same size and shape. In some embodiments, the toe and heel portions have openings therethrough.

The attachment member can be coupled to a center of the arch portion. The toe portion and heel portion can be symmetrical in medial-lateral and fore-aft directions about an axis extending through the attachment member. In some embodiments, the tendon is interchangeable. In some embodiments, the upper attachment element includes downwardly-extending protrusions on opposite sides of the upper attachment element. The protrusions are configured to control a range of motion of the upper attachment element. In some embodiments, the upper attachment element is configured to be rotated to adjust positions of the protrusions.

All of these embodiments are intended to be within the scope of the disclosure herein. These and other embodiments will become readily apparent to those skilled in the art from the following detailed description having reference to the attached figures, the disclosure not being limited to any particular disclosed embodiment(s).

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 illustrates a side view of an example embodiment of a prosthetic board sports foot;

FIG. 2 illustrates a side perspective view of the prosthetic board sports foot of FIG. 1;

FIG. 3 illustrates a top view of the prosthetic board sports foot of FIGS. 1-2;

FIG. 4 illustrates a side perspective view of another example embodiment of a prosthetic board sports foot;

FIG. 5 illustrates a side view of the prosthetic board sports foot of FIG. 4;

FIG. 6 illustrates a top view of the prosthetic board sports foot of FIGS. 4-5;

FIG. 7 illustrates a side view of an example embodiment of a prosthetic board sports foot including an example embodiment of a traction sole;

FIG. 8 illustrates a side perspective view of an example embodiment of a prosthetic board sports foot including an attachment member having an adjustable tendon;

FIG. 9 illustrates a close-up view of the attachment member of FIG. 8;

FIG. 10 illustrates a section view of an example embodiment of a prosthetic board sports foot including an adjustable tendon;

FIG. 11 illustrates a partial exploded view of an example embodiment of a mechanism for adjusting an orientation of an attachment member for a prosthetic board sports foot;

FIG. 12 illustrates a perspective view of another embodiment of a prosthetic board sports foot;

FIG. 13 illustrates a top view of the prosthetic board sports foot of FIG. 12;

FIGS. 14A-14F illustrate another example embodiment of a prosthetic board sports foot;

FIG. 15 illustrates another example embodiment of a prosthetic board sports foot including a traction sole;

FIG. 16 illustrates the prosthetic board sports foot of FIG. 15 without the traction sole;

FIG. 17 illustrates the attachment member of the prosthetic board sports foot of FIGS. 15-16;

FIG. 18 illustrates an alternative embodiment of an attachment member;

FIG. 19 illustrates an example embodiment of a prosthetic board sports foot including the attachment member of FIG. 18;

FIG. 19A illustrates a section view of the prosthetic foot of FIG. 19;

FIG. 19B illustrates another alternative embodiment of an attachment member;

FIG. 20 illustrates the traction sole of the prosthetic board sports foot of FIG. 15; and

FIG. 21 illustrates an example embodiment of a tendon for a prosthetic board sports foot.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

Natural human feet have evolved to be adapted for walking and/or running and have an asymmetric center of effort with the heel portion shorter than the toe portion. The leverage from the toe therefore differs substantially from that of the heel. For board sports (e.g., surfing, windsurfing, kitesurfing, wakeboarding, skateboarding, snowboarding, skiing, etc.) and related activities, this asymmetry is not ideal. In such sports, the user typically uses a symmetric board and must compensate for the asymmetry of the center of effort of the natural foot by varying his or her stance and weight distribution.

The present disclosure provides various examples of prosthetic sport feet and features for prosthetic sport feet. For example, in some embodiments, a prosthetic sport foot can be a prosthetic foot designed for board sports such as surfing, windsurfing, kitesurfing, wakeboarding, skateboarding, snowboarding, skiing, etc., and/or for activities such as sailing or boating. In other embodiments, a prosthetic sport foot can be adapted for use in other sports or for normal use (e.g., walking). Various features as described herein can advantageously improve the stability of the sport feet and help the user maintain balance and/or a desired center of gravity to improve user performance. Prosthetic feet according to the present disclosure can also include features that provide the foot with a high degree of mobility in both the coronal and sagittal planes. The prosthetic feet shown and described herein also advantageously have relatively simple and robust designs to withstand harsh extreme sport environments, for example, saltwater and sandy or rocky terrain.

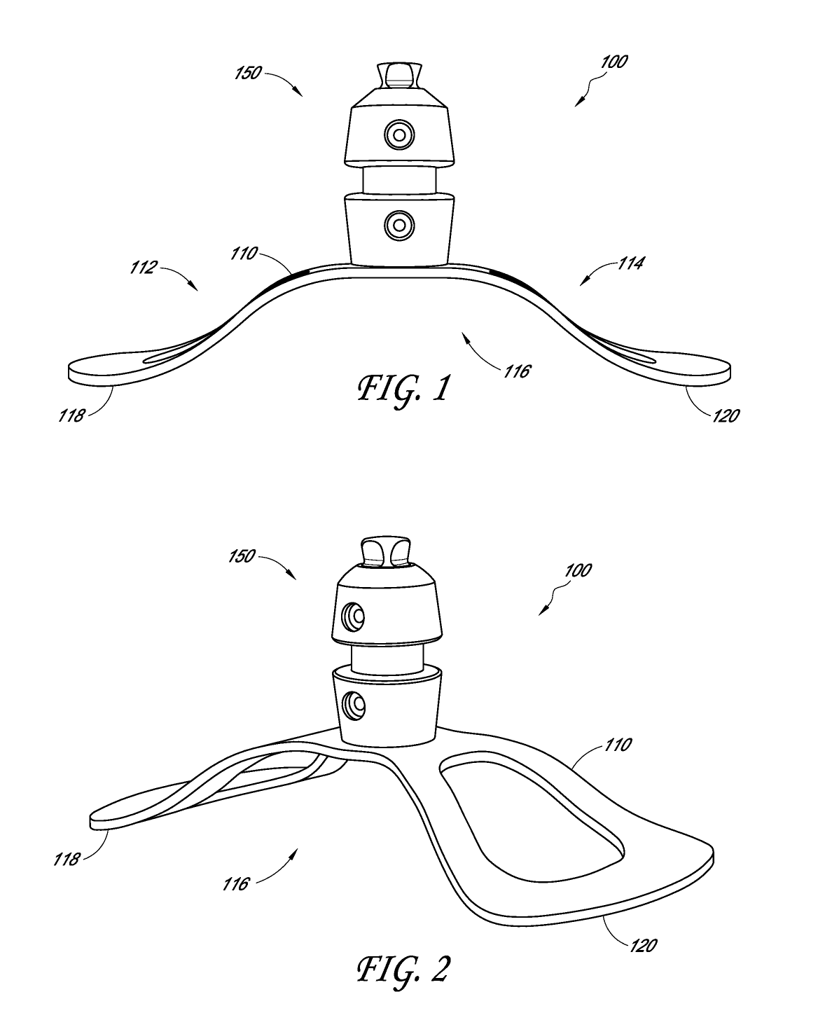

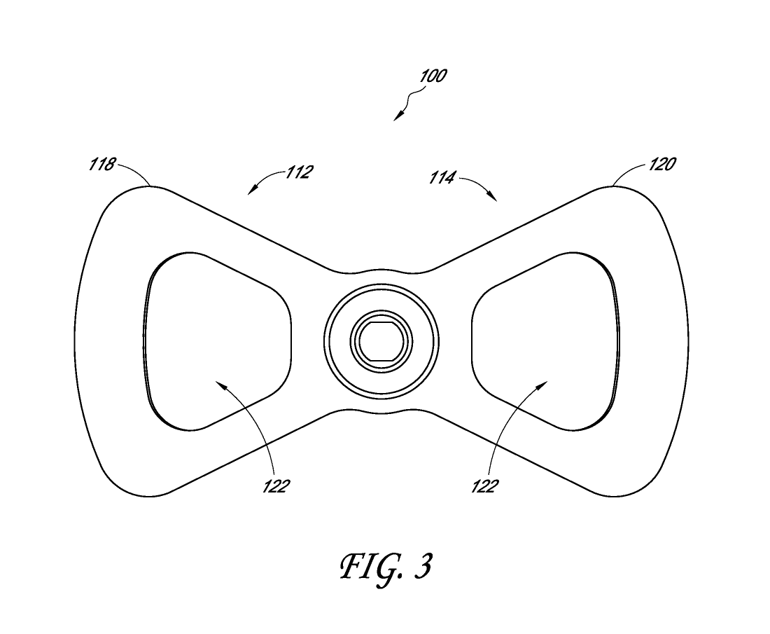

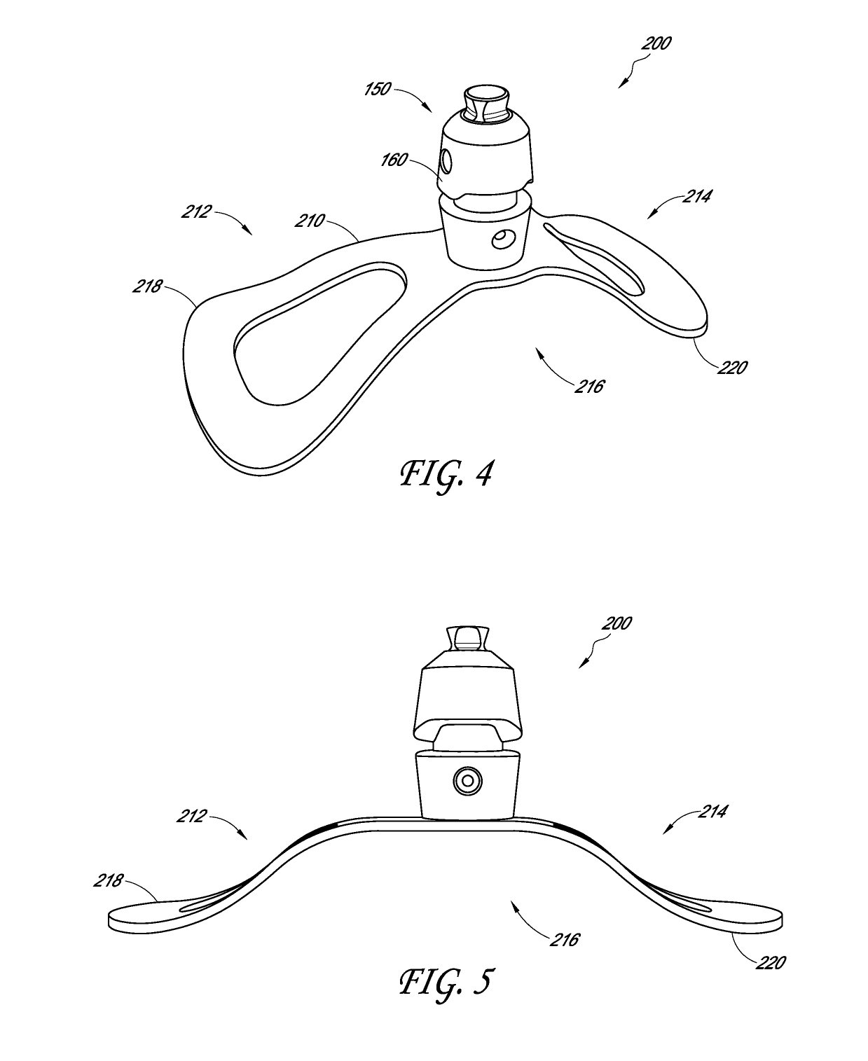

FIGS. 1-3 illustrate an example embodiment of a prosthetic board sports foot 100. As shown, the board sports foot 100 includes a foot member 110 having a toe portion 112, heel portion 114, and arch portion 116 between the toe 112 and heel 114 portions. The arch portion 116 is generally horizontal, and the toe 112 and heel 114 portions extend and curve outwardly and downwardly from the arch portion 116. The toe portion 112 includes a toe ground contacting portion 118 at the front or toe end of the foot member 110, and the heel portion 114 includes a heel ground contacting portion 120 at the rear or heel end of the foot member 110. In the illustrated embodiment, the foot member 110 curves downward from the arch portion 116 to a mid-section of the toe portion 112 and then curves outward to the toe ground contacting portion 118. A transition between the arch portion 116 and the mid-section of the toe portion 112 is defined by a generally upwardly-facing convex portion, and a transition between the mid-section of the toe portion 112 and the toe ground contacting portion 118 is defined by an upwardly-facing concave portion. Similarly, the foot member 110 curves downward from the arch portion 116 to a mid-section of the heel portion 114 and then curves outward to the heel ground contacting portion 120. A transition between the arch portion 116 and the mid-section of the heel portion 114 is defined by a generally upwardly-facing convex portion, and a transition between the mid-section of the heel portion 114 and the heel ground contacting portion 120 is defined by an upwardly-facing concave portion.

The toe 112 and heel 114 portions flare outwardly (in the medial-lateral direction), or increase in width, from a center of the foot member 110 toward the toe ground contacting portion 118 and heel ground contacting portion 120. This advantageously gives the foot 100 a broad profile and an increased surface area in contact with the board during use, which improves stability and increases the friction force with the board to improve grip. In some embodiments, the width of the toe 112 and/or heel 114 portions is greater than the width of a natural human foot. In some such embodiments, the width of the toe 112 and/or heel 114 portions is up to about 3 times wider, for example, about 1.5-2 times wider or about 2-3 times wider, than a natural human foot.

The toe ground contacting portion 118 and heel ground contacting portion 120 are spaced relatively far from each other to improve leverage. In some embodiments, a length of the foot member 110 is similar to or substantially the same as the user's sound foot (or similar to or substantially the same as a length the user's natural foot would be). A length within the range of natural human feet advantageously allows the foot 100 to be used with standard or typical boards available, which are often made with widths corresponding to natural human foot lengths. A thickness of the foot member 110 can be selected based on the particular user or intended use for the foot 100. The large arch portion 116 advantageously provides suspension, stability, and balance. The large arch portion 116 can also help ensure the toe ground contacting portion 118 and heel ground contacting portion 120 maintain contact with the board (or ground) throughout a wide range of use as the direction of force on the foot changes. The illustrated foot member 110 is monolithic and made of a fiber material (e.g., carbon fiber). However, in other embodiments, the foot member 110 can be modular and/or made of other suitable materials. The fiber or other material and/or a layup sequence of the fiber or other material can be selected based on the particular user or intended use for the foot 100. In the illustrated embodiment, the toe 112 and heel 114 portions include holes or openings 122. The openings can advantageously reduce the overall weight of the foot member 110. The openings can also advantageously provide the foot with greater flexibility and allow for a greater range of twisting. For example, if the user is turning the board and places more weight or pressure on a particular part of the foot 100, for example, a “big toe” region, the rest of the foot member 110 is able to twist to allow the full toe ground contacting portion 118 and heel ground contacting portion 120 to maintain contact with the board. Additionally, for board sports where the user is in the water at least some of the time (e.g., surfing where the user paddles out into the water or may be swimming after falling off the board), the openings can help reduce the drag of the foot member 110 in the water.

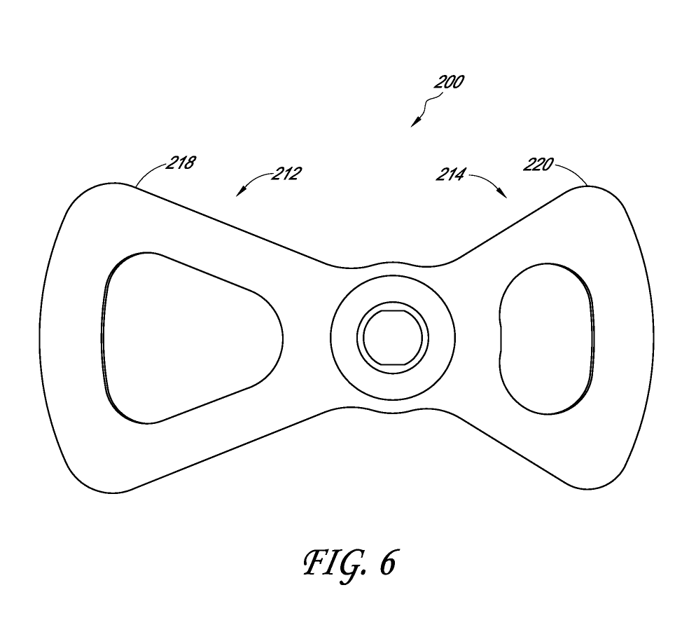

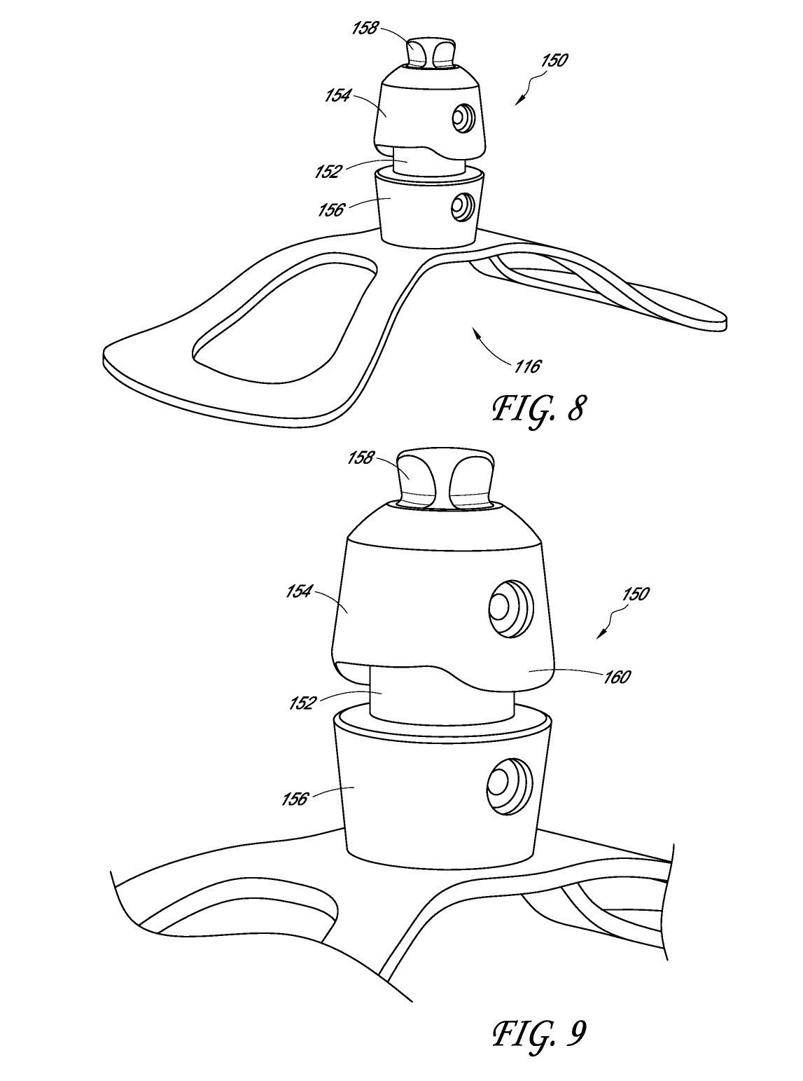

In the embodiment of FIGS. 1-3, the board sports foot 100 is symmetric in both medial-lateral and anterior-posterior directions, which can advantageously allow the user to compensate for and use a symmetric board more easily. However, in other embodiments, a board sports foot can be asymmetric. For example, FIGS. 4-6 illustrate an example embodiment of a board sports foot 200 having a foot member 210 that is asymmetric in the anterior-posterior direction. As shown, the toe portion 212 of board sports foot 200 is longer than the heel portion 214. This shifts the center of gravity of the foot, which can advantageously provide more springiness in the toe portion 212 during use. In some embodiments, a foot member, such as foot member 110, 210 or a foot member in another embodiment of a prosthetic board sports foot, can be asymmetric in the medial-lateral direction. For example, the medial side of the toe portion and/or heel portion can be longer or shorter than the lateral side. In the illustrated embodiment, the toe ground contacting portion 218 and heel ground contacting portion 220 have equal or approximately equal widths. However, in other embodiments, the toe portion 212 and toe ground contacting portion 218 can be wider or narrower than the heel portion 214 and heel ground contacting portion 220.

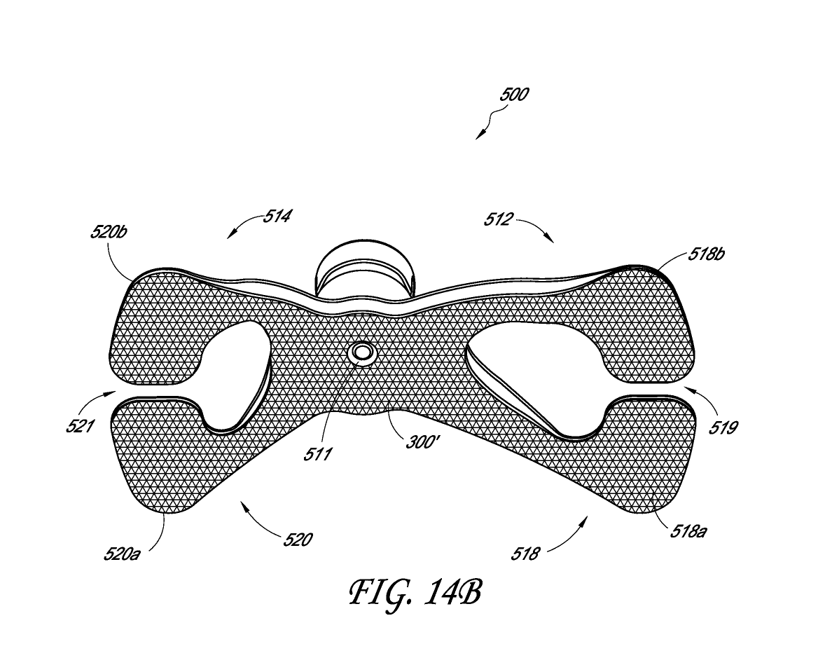

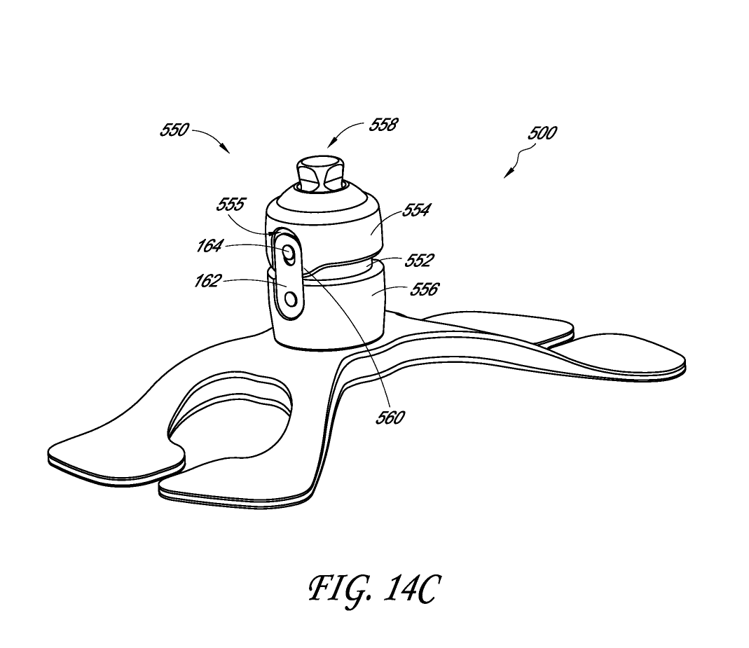

In the embodiments of FIGS. 1-6, the foot members 110, 210 include continuous toe ground contacting portions 118, 218 and heel ground contacting portions 120, 220. FIGS. 12-13 illustrate another example embodiment of a board sports foot 400 having a foot member 410 in which each of the toe ground contacting portion 418 and heel ground contacting portion 420 is discontinuous. As shown, the toe ground contacting portion 418 is separated into two portions 418a, 418b by a gap 419. Similarly, the heel ground contacting portion 420 is separated into two portions 420a, 420b by a gap 421. The gaps 419, 421 are positioned in the sagittal plane and separate each of the toe 412 and heel 414 portions into medial and lateral blades. The gaps 419, 421 can allow the medial and lateral blades or portions of the toe 412 and heel 414 portions to flex at least somewhat independently of each other. This can allow the foot member 410 to better adapt to a variety of boards or ground surfaces and to a variety of user movements. Although in the illustrated embodiment both the toe ground contacting portion 418 and heel ground contacting portion 420 are separated by gaps 419, 421, respectively, in other embodiments, only one of the toe ground contacting portion 418 and heel ground contacting portion 420 may be separated by a split and the other may be continuous.

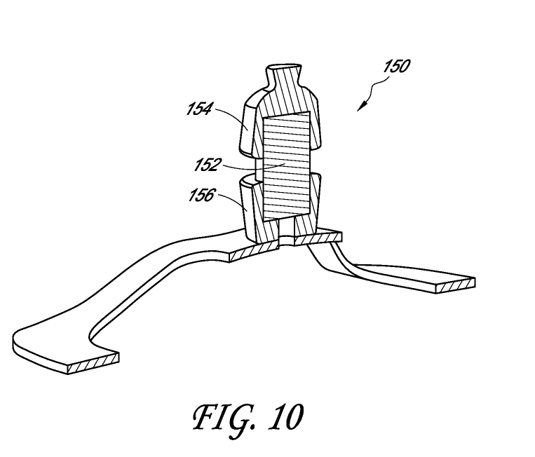

FIG. 7 illustrates an example embodiment of a traction sole 300 for use with board sports feet, such as the board sports feet 100, 200 shown and described herein. The traction sole 300 can advantageously help prevent or inhibit slipping on the board or other surface. In the illustrated embodiment, the traction sole 300 extends or stretches across the bottom of the foot member 210. The traction sole 300 is attached, either removably or permanently, to the toe ground contacting portion 118, 218 and the heel ground contacting portion 120, 220, but is not attached to the arch portion 116, 216. The portion of the traction sole 300 spanning the arch portion 116, 216 can conform to the board or ground surface in use.

In other embodiments, the traction sole is attached, either removably or permanently, to only the toe ground contacting portion 118, 218 and/or heel ground contacting portion 120, 220 and does not span the arch portion 116, 216. For example, the traction sole can include two separate components, one attached to the toe ground contacting portion 118, 218 and the other attached to the heel ground contacting portion 120, 220. In yet other embodiments, the traction sole extends across and is attached, either removably or permanently, to the entire lower surface of the foot member 110, 210. In some such embodiments, the traction sole may also wrap around the outer edges or perimeter of the foot member 110, 210 to or toward the upper surface of the foot member 110, 210. This can advantageously help protect the foot member 110, 210 and/or board or other equipment from damage or wear and/or help protect the user from injury, for example, if the user were to strike his or her sound leg with an edge of the foot member 110, 210. In some embodiments, the traction sole is designed to correspond to or fit into a binding on the board, e.g., such as bindings on snowboards, skis, and wakeboards.

In some embodiments, the toe 312 and heel 314 ends of the traction sole 300 can include cavities or slots that removably receive the toe and heel ends of the foot member 110, 210. The toe 312 and heel 314 ends of the traction sole 300 can wrap around at least a portion of the toe ground contacting portion 118, 218 and heel ground contacting portion 120, 220, respectively, of the foot member 110, 210 to secure the traction sole 300 to the foot member 110, 210. In some embodiments, the traction sole 300 is secured to the foot member 110, 210 via clips or another releasable mechanism. Alternatively, the traction sole 300 can be attached to the foot member 110, 210 with an adhesive, such as a contact adhesive or epoxy.

The traction sole can be made of a material that has elastic properties and/or is capable of stretching. Such a material advantageously allows the traction sole to cling tightly to the board or ground surface in use. In some embodiments, the traction sole, or portions thereof, are made of rubber, silicone, etc. or combinations of rubber, silicone, and/or other such materials.

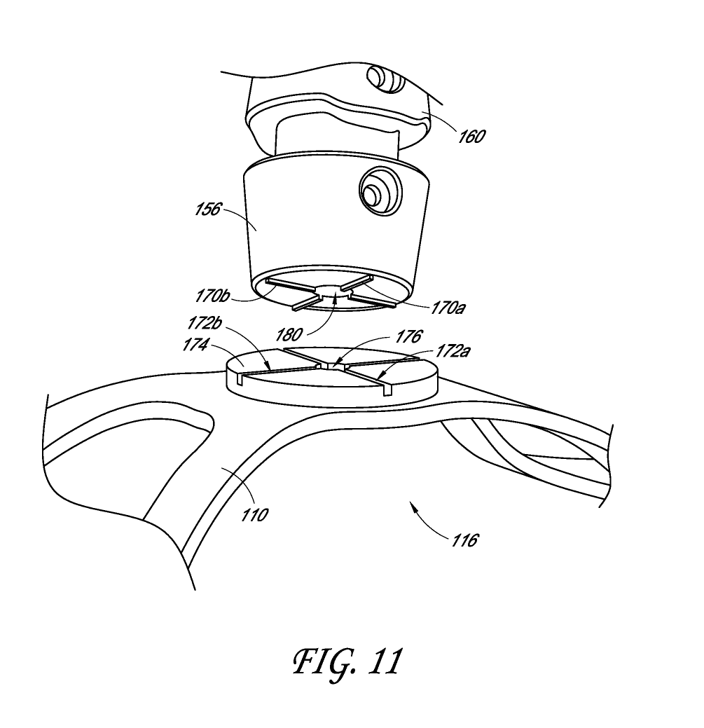

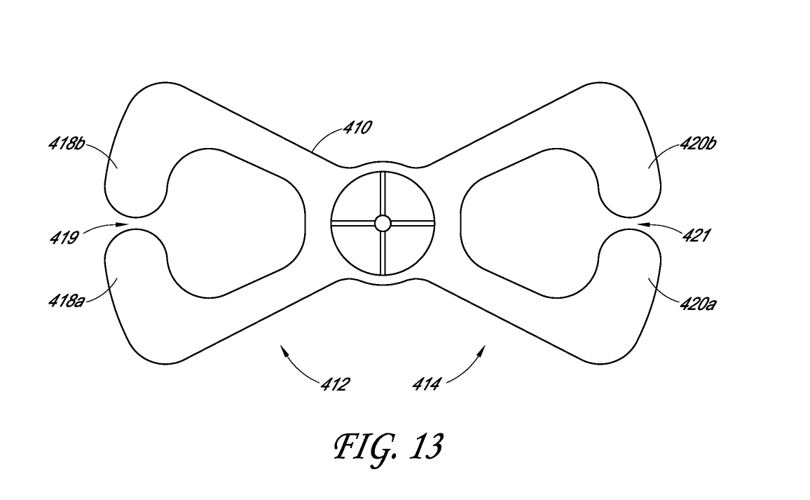

The board sports foot 100, 200 can also include an ankle or attachment member 150. The attachment member 150 is designed to allow for flexibility over an initial short range of movement and then to resist flexion outside of that range to advantageously transfer leverage to the toe portion 112, 212 or heel portion 114, 214. As shown in FIGS. 8-10, the attachment member 150 includes a tendon 152 and upper 154 and lower 156 sockets or attachment elements. The upper socket 154 includes a connector, such as pyramid connector 158, for coupling the board sports foot 100, 200 to the user's residual limb or another prosthetic component (e.g., pylon). The lower socket 156 is coupled to the foot member 110, 210. In the illustrated embodiments, the lower socket 156 is coupled to a midpoint of the arch portion 116, 216. In some embodiments, the attachment member 150, for example, the lower socket 156 of the attachment member 150 is coupled to the foot member 110, 210 via bolts, fasteners, or the like.

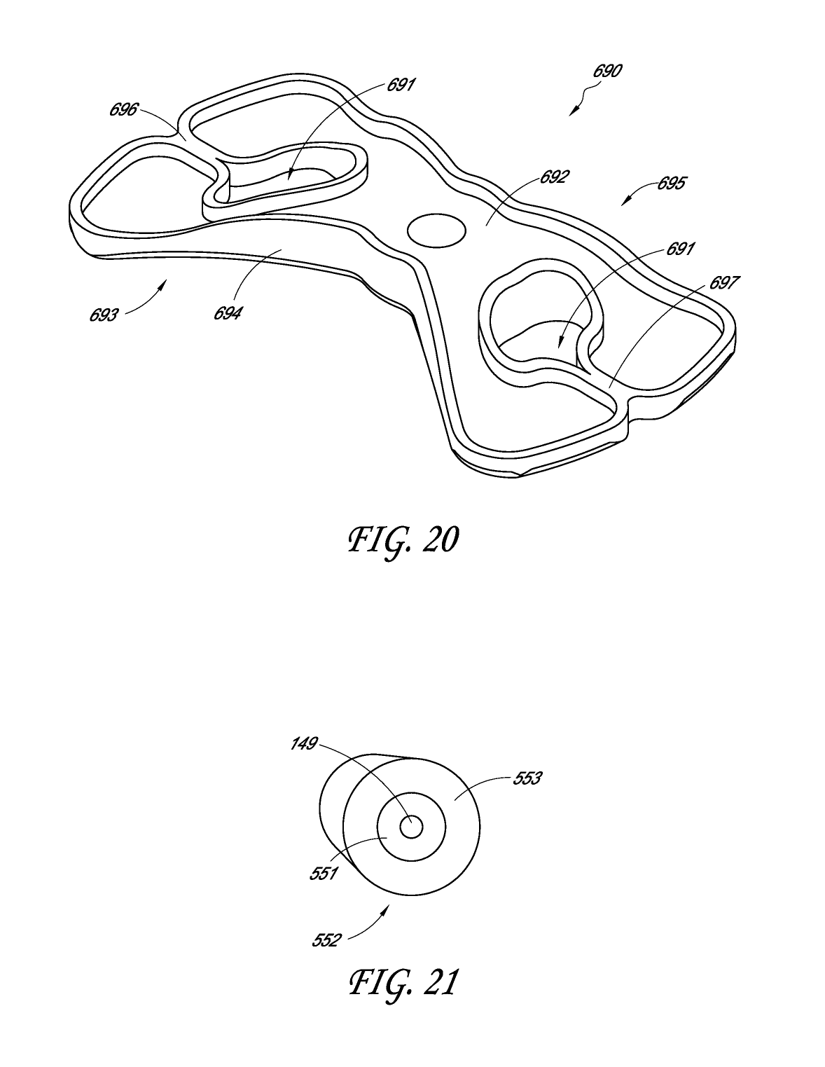

The tendon 152 can be interchangeable, and tendons 152 having varying stiffnesses can be provided. In some embodiments, the tendon 152 can be interchanged by the user. This advantageously allows the foot to be adjusted and tuned relatively easily by the user to suit the particular preferences and intended use(s) of the individual user. In some embodiments, the tendon(s) 152 can be made of various rubber and/or elastic materials. In some embodiments, the tendon(s) 152 includes laminated sections of different stiffnesses. For example, opposite sides of the tendon 152 can be stiffer than the middle of the tendon 152. This can advantageously provide the tendon 152 with variable or different stiffness in one plane (e.g., in a medial/lateral or fore/aft direction) compared to another plane. In some embodiments, the tendon 152 has an outer shell and an inner core. In some such embodiments, the outer shell is made of a material of a relatively high stiffness, and the inner core is made of a material (e.g., rubber) having a relatively lower stiffness. However, in other embodiments, the outer shell can be made of a material having a lower stiffness than the outer core. The inner core can be removable and interchangeable. This can advantageously allow the user or a prosthetist to select an inner core to customize the overall stiffness of the tendon 152. In some embodiments, the tendon 152 can include a wire or tether extending through the middle of the tendon 152. For example, the example embodiment of a tendon 552 shown in FIG. 21 includes a wire 149. The wire or tether is coupled or attached to the upper 154 and/or lower 156 sockets or attachment elements such that if the tendon 152 breaks during use, the wire or tether can inhibit or prevent the user from losing the foot.

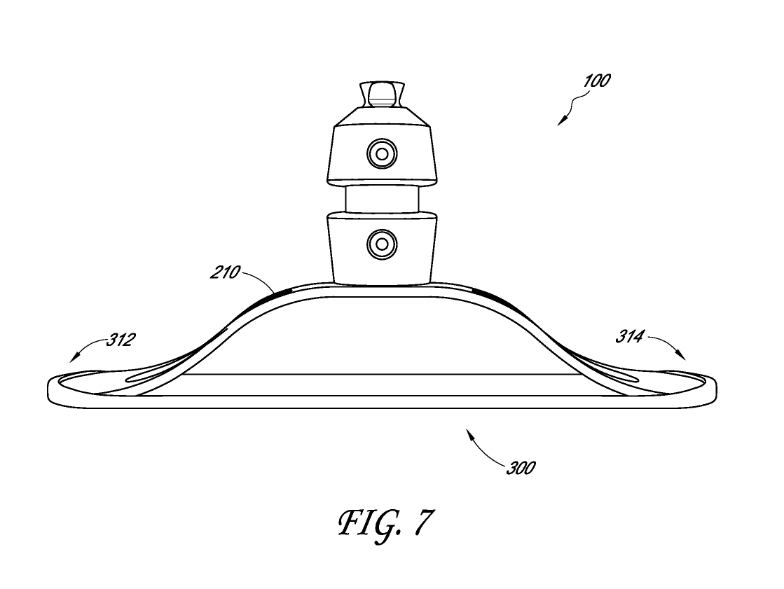

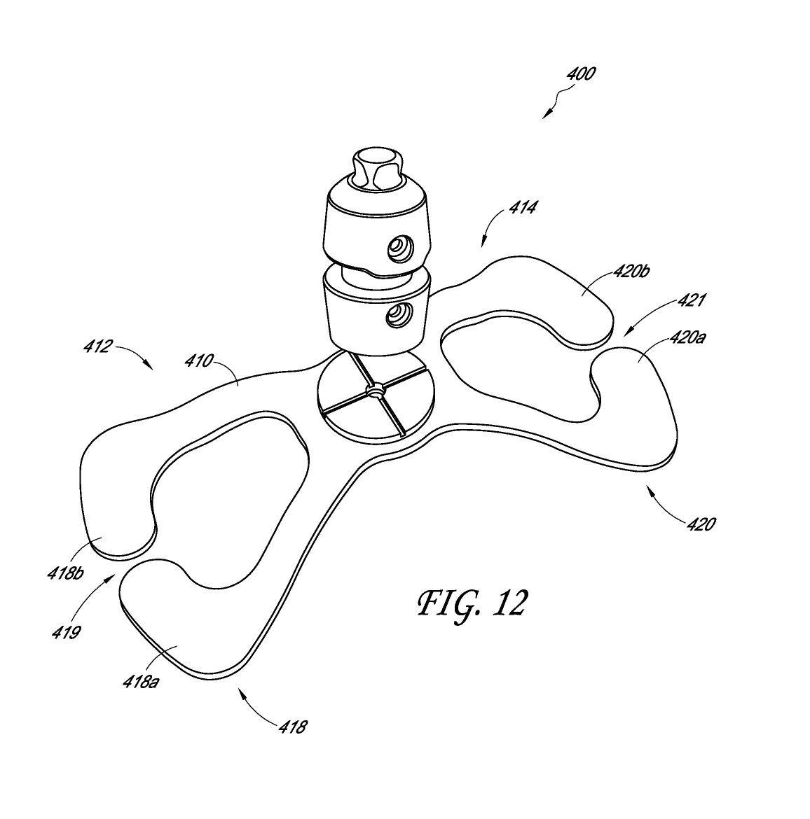

In some embodiments, one or both of the upper 154 and lower 156 sockets or attachment elements are asymmetric. For example, as shown in FIGS. 4 and 5, the upper attachment element 154 includes protruding ridges or downwardly extending protrusions 160 at the front and back. The ridges 160 can advantageously guide or limit the range of motion of the upper attachment element 154 in the forward and back direction. In the embodiment shown in FIGS. 8 and 9, the upper attachment element 154 includes protruding ridges 160 on the medial and lateral sides. These ridges 160 can guide or limit the range of motion of the upper attachment element 154 in the medial-lateral direction. In other embodiments, the upper attachment element 154 can include a protruding ridge 160 on only the front, back, medial, or lateral side or protruding ridges 160 on the front, back, medial, and lateral sides. Including only a single protruding ridge 160 allows movement to be controlled in a particular direction (e.g., either forward, backward, medial, or lateral). Various combinations of protruding ridges 160 at various positions are also possible. For example, the upper attachment element 154 can include protruding ridges 160 on adjacent sides or spaced approximately 90° from each other to control or limit movement in one direction in each plane while allowing more free movement in the other direction in each plane.

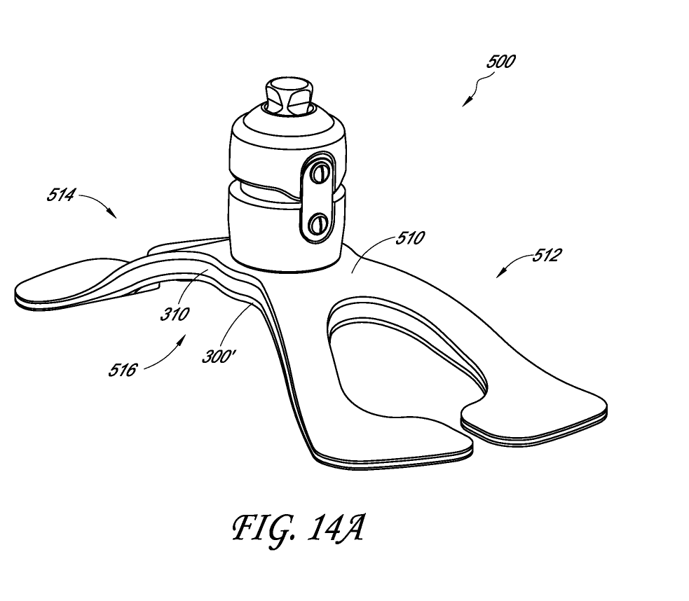

In some embodiments, the upper attachment element 154 can be rotated to change the position of the protruding ridge(s) 160. This can advantageously allow the attachment element 154 to be adjusted for different activities, conditions, or preferences. In some such embodiments, the upper attachment element 154 can be rotated by the user so that the user can tune the attachment member 150 as desired. In some embodiments, the entire ankle member 150 can be rotated. In some such embodiments, the lower attachment element 156 includes two protrusions or ridges 170a, 170b extending along a bottom surface of the lower attachment element 156, for example as shown in FIG. 11. One of the protrusions extends across the bottom of the lower attachment element 156 in the sagittal plane, and the other extends across the bottom in the coronal plane. The protrusions intersect each other at about a 90° angle at the center of the lower attachment element 156. In the illustrated embodiment, the center of the lower attachment element 156 includes a hole 180 that can receive a fastener (e.g., bolt, screw, or the like) for securing the lower attachment element 156 to the foot member 110. As shown, the hole 180 can divide each of the ridges 170a, 170b into two halves.

A top surface of the arch portion 116 of the foot member 110 includes corresponding grooves 172a, 172b that receive the protrusions 170a, 170b. In the illustrated embodiment, the grooves 172a, 172b are formed in a member 174 coupled to the top surface of the arch portion 116. As shown, the member, such as a disk, 174 can include a hole 176 that can receive the fastener for securing the lower attachment element 156 to the foot member 110. The fastener can also secure the disk 174 to the foot member 110. In the illustrated embodiment, the hole 176 divides each of the grooves 172a, 172b into two halves. The disk 174 can also or alternatively be coupled to the foot member 110 via an adhesive. In other embodiments, the foot does not include a disk 174, and the grooves 172a, 172b can be formed directly in the foot member 110. To rotate the position of the protruding ridge(s) 160, the user can uncouple (e.g., unscrew) the ankle member 150 from the foot member 110, rotate the ankle member 150 90°, and recouple the ankle member 150 to the foot member 110. The protrusions on the lower attachment element 156 and corresponding grooves on the foot member 110 can help the user orient the ankle member 150. Though the embodiment in FIG. 11 shows only two ridges 170a, 170b generally perpendicular to each other and two corresponding grooves 172a, 172b, the system can have more than two ridges and corresponding grooves at various angles relative to each other. Furthermore, in some embodiments, the bottom surface of the lower attachment element 156 can include grooves instead of protrusions, and the top surface of the arch portion of the foot member 110 or a disk 174 coupled to the top surface of the arch portion 116 can include corresponding protrusions or ridges instead of grooves.

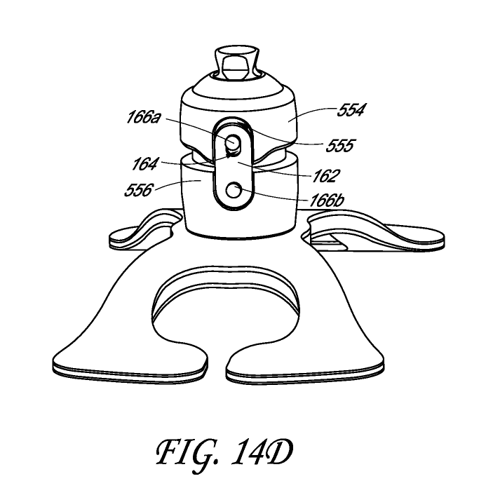

FIGS. 14A-14F illustrate another example embodiment of a prosthetic board sports foot 500. In the illustrated embodiment, board sports foot 500 has a foot member 510 that is asymmetric in the anterior-posterior direction with a toe portion 512 that is longer than a heel portion 514 as shown in FIG. 14B. In other embodiments, the foot member 510 can be symmetric in the anterior-posterior direction. The toe ground contacting portion 518 is separated into two portions 518a, 518b by a gap 519, and the heel ground contacting portion 520 is separated into two portions 520a, 520b by a gap 521. The gaps 519, 521 are positioned in the sagittal plane and separate each of the toe 512 and heel 514 portions into medial and lateral blades. In the illustrated embodiment, the foot member 510 is symmetric in the medial-lateral direction. In other embodiments, the foot member 510 can be asymmetric in the medial-lateral direction. For example, the medial blade of the toe portion 512 and/or heel portion 514 can be longer or shorter than the lateral blade.

Board sports foot 500 includes a traction sole 300′ that extends across the entire or substantially the entire length of the bottom of the foot member 510. In one embodiment, the traction sole 300′ is a separate layer attached to the bottom of the board sports foot 500. In another embodiment, the traction sole 300′ forms part of a monolithic foot member 510 (e.g., the traction sole 300′ is provided by a roughened surface formed on a bottom of the foot member 510 during or following manufacture of the foot member 510). In some embodiments, only the portions of the foot member (e.g., the “toe” portions and “heel” portions) that contact a ground surface include the traction sole 300′.

In the illustrated embodiment, the board sports foot 500 also includes an intermediate layer 310 between the foot member 510 and traction sole 300′ along the arch portion 516 and, optionally, at least a portion of the toe portion 512 and/or heel portion 514. The intermediate layer 310 increases the build height from the foot member 510 to the traction sole 300′. An increased build height can advantageously allow the bolt or other fastener coupling the ankle member 550 to the foot member 510, such as bolt 511 shown in FIG. 14B, to be embedded within the intermediate layer 310 so that the fastener does not contact the board or other support surface in use. The thickness or build height of the intermediate layer 310 can be selected or designed based on, for example, the weight of the user and/or the activity the board sports foot will be used for (e.g., surfing, snowboarding, skiing, etc.). The impact placed on the foot can vary during these different activities and can vary with the user's weight. The thickness or build height of the intermediate layer 310 can therefore be selected or designed such that when a certain amount of pressure or force is placed on the foot, the arch portion 516 is pressed downward or toward the support surface enough such that the traction sole 300′ (or bottom of the intermediate layer 310 in the absence of a traction sole 300′ in the arch portion 516) may contact the board or other support surface. This can advantageously allow the portion of the traction sole 300′ in the arch portion 516 to contact the board or support surface to provide additional traction or friction and reduce the likelihood of the foot slipping. The contact of the intermediate layer 310 with the board or support surface during use can also help absorb forces during use to inhibit the foot member 510 from being stressed and/or deforming beyond a critical point and breaking. The intermediate layer 310 can be monolithic or formed of multiple layers of material having the same or different stiffnesses.

In the illustrated embodiment, the intermediate layer 310 is made of EVA foam. Other materials are also possible, such as, for example, polyurethane or polystyrene (e.g., polyurethane or polystyrene foam), such as extruded polystyrene or expanded polystyrene, neoprene, or acrylamide rubber. In some embodiments, the intermediate layer 310 can be integrally formed or molded with the foot member 510. For example, the intermediate layer 310 can be inserted between layers of the foot member 510 during manufacture (e.g., between top and bottom layers of a layup sequence forming the foot member). Alternatively, the intermediate layer 310 can be molded, adhered, or otherwise attached to the foot member 510 post-production. In some embodiments, the intermediate layer 310 can be hollow. In some embodiments, the intermediate layer 310 and/or traction sole 300′ can be removably attached or coupled to the foot member 510. For example, the intermediate layer 310 and/or traction sole 300′ can be removably coupled to the foot member 510 via clips or other attachment mechanism. In some such embodiments, a user can select an intermediate layer 310 and/or traction sole 300′ from a variety of options designed for different activities and can interchange the intermediate layer 310 and/or traction sole 300′ as desired for different activities.

Board sports foot 500 includes an ankle member 550 that includes an upper attachment element 554, a lower attachment element 556, and a tendon 552. The upper attachment element 554 includes a connector, such as pyramid connector 558, for coupling the board sports foot 500 to the user's residual limb or another prosthetic component (e.g., pylon). In the illustrated embodiment, the ankle member 550 includes inserts 162 coupled to and extending between the upper attachment element 554 and the lower attachment element 556 along the front and/or back of the ankle member 550. As shown in FIGS. 14C and 14D, the front and/or back of the upper attachment element 554 includes a recess 555 extending upward from a distal end of the upper attachment element 554. The recess 555 receives a proximal or upper portion of the insert 162. The insert 162 includes an elongated hole 164 in the proximal or upper portion. A pin 166a extends through the hole 164 and into engagement with the recess 555. As shown in FIGS. 14E and 14F, a fastener 168a, such as a bolt, screw, or other fastener, can be coupled to or about the pin 166a to secure the insert 162 to the upper attachment element 554. The lower attachment element 556 can also include a recess extending downward from a proximal end of the lower attachment element 556 to receive a distal or lower portion of the insert 162. A second pin 166b extends through the distal or lower portion of the insert 162 and into engagement with the lower attachment element 556, and a second fastener 168b can be coupled to or about the second pin 166b to secure the insert 162 to the lower attachment element 556. In some embodiments including inserts 162 along both the front and back of the ankle member 550, the pins 166a, 166b can extend through the tendon 552 and both of the inserts 162. The insert 162 can be made of metal or other appropriate materials. In some embodiments, the insert 162 is integrally formed or molded with the upper attachment element 554 and/or lower attachment element 556.

The elongated hole 164 and recess 555 allow the upper attachment element 554 to move relative to the insert 162 within a given range (e.g., the upper attachment element 554 can tilt forward or backward until the top of the insert 162 abuts the top end of the recess 555 and the pin 166a abuts the bottom of the elongated hole 164) while the insert 162 remains fixed relative to the lower attachment element 556). This allows the upper attachment element 554 to compress the tendon 552 in the sagittal plane but inhibits (e.g., prevents) the upper attachment element 554 from stretching the tendon 552 in the sagittal plane (e.g., because the pin 166a abuts the top of the elongated hole 164), thereby substantially removing a degree of freedom in the tendon 552 for motion in the sagittal plane. The upper attachment element 554 can move relative to the lower attachment element 556 in a medial-lateral direction due to motion of the upper attachment element 554 and/or insert 162 about one or both of the pins 166a, 166b and/or motion of the insert 162 relative to the recess 555. The tendon 552 is therefore relatively less flexible in the sagittal plane and relatively more flexible in the coronal plane. As shown in FIG. 14C, the upper attachment element 554 can also include downwardly extending protrusions 560 at the front and/or back to advantageously guide or limit the range of motion of the upper attachment element 554 relative to the lower attachment element 556 in the forward and backward direction. In some embodiments, a gap between a bottom of the protrusion(s) 560 and the top of the lower attachment element 556 is smaller than a gap between the top of the insert 162 and top end of the recess 555 and/or a gap between the pin 166a and the bottom of the elongated hole 164 such that the protrusion(s) 560 abuts the lower attachment element 556 in compression before the top of the insert 162 abuts the top end of the recess 555 and/or the pin 166a abuts the bottom of the elongated hole 164. In such an embodiment, the protrusion(s) 560 provide the primary mechanism for limiting compression of the tendon 552 in the sagittal plane while the insert 162 provides a mechanism for inhibiting stretching of the tendon 552 in the sagittal plane. This can advantageously reduce the amount of stress placed on the insert 162.

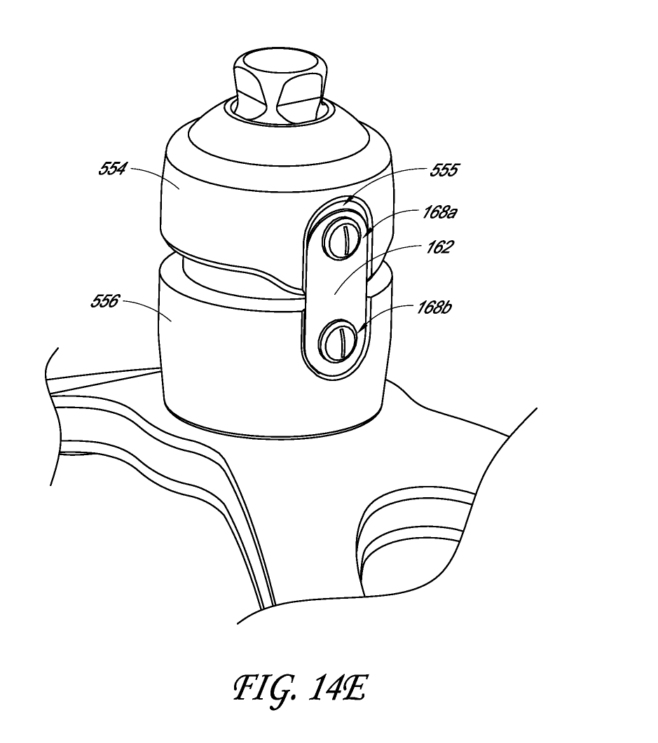

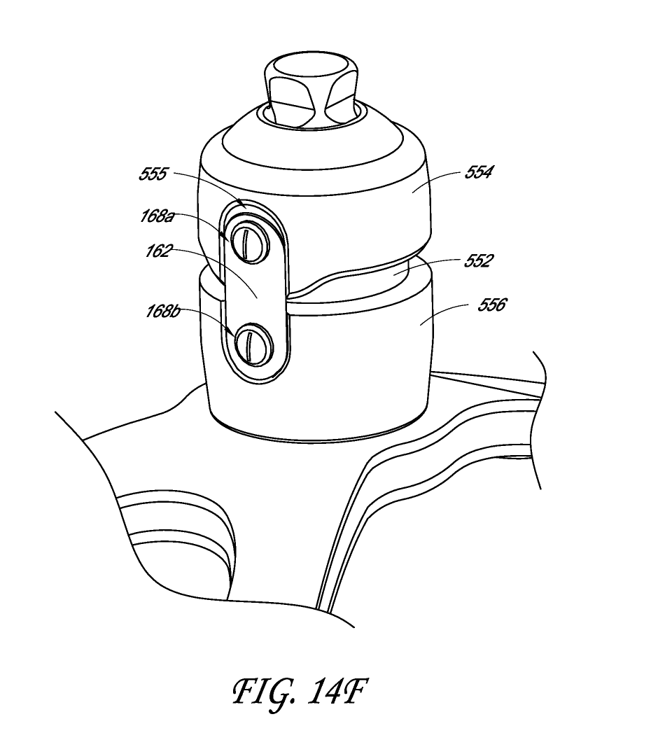

FIGS. 15-16 illustrate another example embodiment of a prosthetic board sports foot 600. Board sports foot 600 can include various features similar to those of board sports foot 500. As shown, board sports foot 600 includes a foot member 610 and an ankle member 650. Foot member 610 can be similar to foot member 510. For example, in the illustrated embodiment, foot member 610 is asymmetric in the anterior-posterior direction with a toe portion 612 that is longer than a heel portion 614, toe ground contacting portion 618 is separated into two portions 618a, 618b by a gap 619, and heel ground contacting portion 620 is separated into two portions 620a, 620b by a gap 621. In other embodiments, however, the toe and heel portions can be symmetrical and/or the toe and/or heel ground contacting portions can be continuous (e.g., not separated into two portions by a gap).

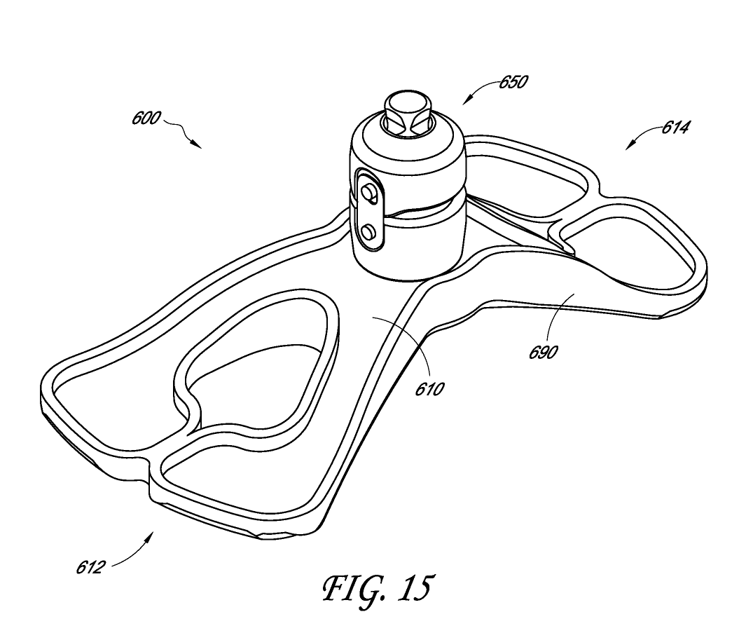

Ankle member 650 can be similar to ankle member 550. For example, as shown in FIG. 17, ankle member 650 includes an upper attachment element 654 including a connector, such as a pyramid connector 758, a lower attachment element 656, a tendon 652, and inserts 162 coupled to and extending between the upper attachment element 654 and the lower attachment element 656 (e.g., within recesses) along the front and/or back of the ankle member 650. The upper attachment element 654 can also include downwardly extending protrusions 660 at the front and/or back to advantageously guide or limit the range of motion of the upper attachment element 654 relative to the lower attachment element 656 in the forward and backward direction. In some embodiments, the lower attachment element 656 includes two protrusions or ridges 670a, 670b extending along a bottom surface of the lower attachment element 656 that can be received in or engage corresponding grooves in a top surface of the arch portion of the foot member 610 or a member coupled to the top surface of the arch portion. In the illustrated embodiment, one of the protrusions extends across the bottom of the lower attachment element 656 in the sagittal plane, the other extends across the bottom in the coronal plane, and the protrusions 670a, 670b intersect each other at about a 90° angle at the center of the lower attachment element 656. In the illustrated embodiment, the center of the lower attachment element 656 includes a hole 680 that can receive a fastener (e.g., bolt, screw, or the like) for securing the lower attachment element 656 to the foot member 610. As shown, the hole 680 can divide each of the ridges 670a, 670b into two halves. Though the illustrated embodiment shows only two ridges 670a, 670b generally perpendicular to each other to engage two corresponding grooves in the foot member 610 or a disk coupled to the foot member 610, the system can have more than two ridges and corresponding grooves at various angles relative to each other. Furthermore, in some embodiments, the bottom surface of the lower attachment element 656 can include grooves instead of protrusions, and the top surface of the arch portion of the foot member 610 or a disk coupled to the top surface of the arch portion can include corresponding protrusions or ridges instead of grooves.

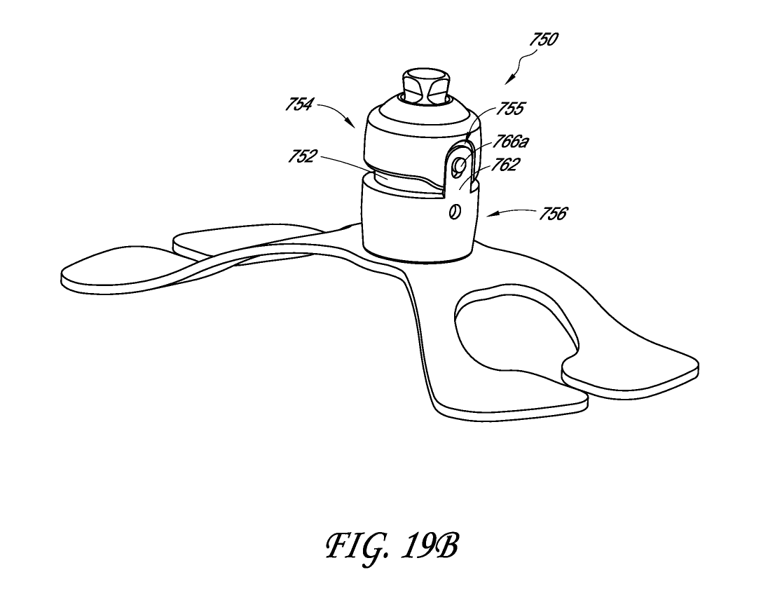

As shown in FIG. 15, board sports foot 600 can also include a traction sole 690, shown in FIG. 20. In the illustrated embodiment, the traction sole 690 can be removably coupled to the foot member 610. In other embodiments, the traction sole 690 can be permanently coupled to the foot member 610. As shown in FIG. 20, the traction sole 690 has a base 692 and a sidewall 694 extending upwardly from the base 692. When the traction sole 690 is coupled to the foot member 610, the base 692 extends along the bottom surface of the foot member 610 and the sidewall 694 extends along the edges or perimeter of the foot member 610 so that when the foot member 610 is coupled to the traction sole 690, the foot member 610 is disposed in a recess defined by the base 692 and sidewall 694. The traction sole 690 can be made of a durable rubber material. In some embodiments, the traction sole 690 is made of EVA. The traction sole 690 can be somewhat flexible such that the traction sole 690 can be stretched slightly to be coupled to and removed from the foot member 610.

As shown, the traction sole 690 generally corresponds in size and shape to the foot member 610. In the illustrated embodiment, the traction sole 690 therefore is asymmetric in the anterior-posterior direction, includes holes or openings 691 in the toe portion 693 and heel portion 695 that correspond to holes or openings 122 in the foot member, and accommodates the two portions 618a, 618b of the toe ground contacting portion 618 and the two portions 620a, 620b of the heel ground contacting portion 620. In the illustrated embodiment, the traction sole 690 includes a partition 696 that is disposed between the two portions 618a, 618b of the toe ground contacting portion 618 and a partition 697 that is disposed between the two portions 620a, 620b of the heel ground contacting portion 620 when the traction sole 690 is coupled to the foot member 610. In other embodiments, the toe portion 693 and heel portion 695 of the traction sole 690 can include gaps rather than partitions 696, 697 corresponding to the gaps 619, 621 between the portions 618a, 618b of the toe ground contacting portion 618 and portions 620a, 620b of the heel ground contacting portion 620. In some embodiments, the traction sole 690 has a continuous toe ground contacting portion and/or heel ground contacting portion (i.e., the toe and/or heel ground contacting ports are not divided into medial and lateral portions by partitions 696, 697 or gaps). In other embodiments, a traction sole according to the present disclosure can be used with other foot members as described herein.

The sidewall 694 can advantageously help secure the traction sole 690 to the foot member 610. The sidewall 694 can also help protect the edges of the foot member 610 from damage that may be caused by the board or environment during use and/or protect the board or surrounding objects from damage that may be caused by the edges of the foot member 610. In some embodiments, the sidewall 694 can extend above the top surface of the foot member 610. In other embodiments, the sidewall 694 can be substantially flush with or substantially the same height as the top surface of the foot member 610. As described herein, in some embodiments the thickness of the foot member 610, and therefore the height of the sidewall 694, can be selected based on the particular user and/or intended use of the foot.

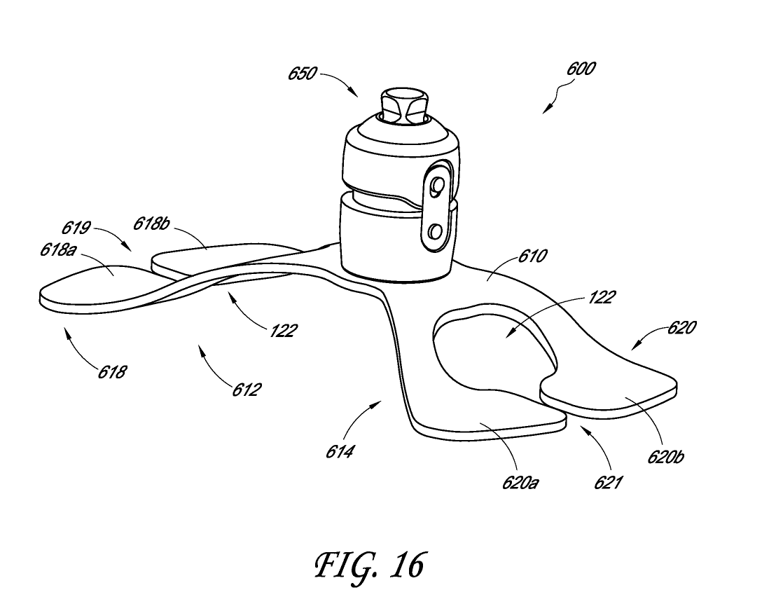

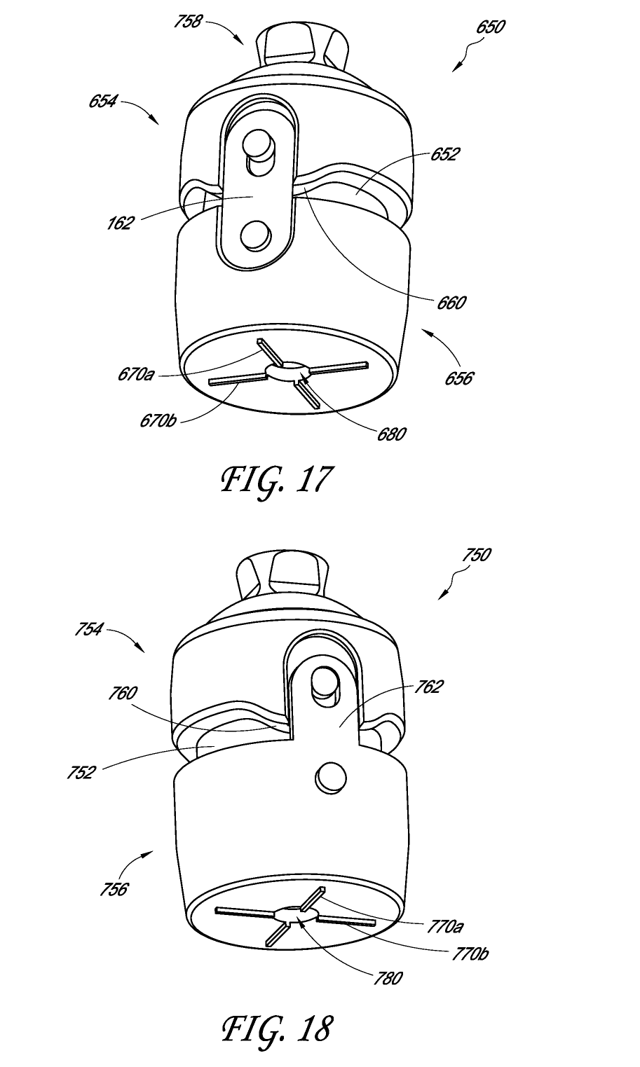

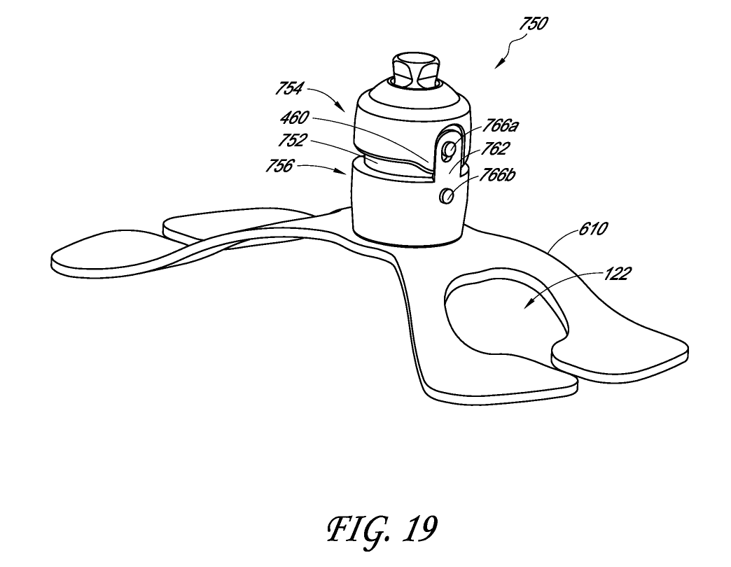

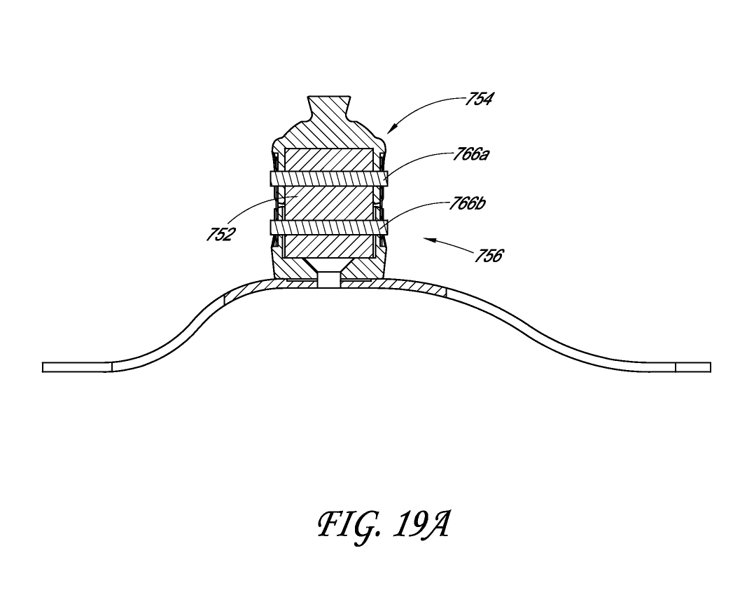

FIGS. 18 and 19 illustrate another example embodiment of an ankle member 750 including an upper attachment element 754, lower attachment element 756, and a tendon 752. The ankle member 750 can be coupled to a foot member as described herein, for example, foot member 610 as shown in FIG. 19. Ankle member 750 includes an insert 762 that is integrally formed with the lower attachment element 756. In other embodiments, the insert 762 can be integrally formed with the upper attachment element 754. In the embodiment illustrated in FIGS. 18-19, the ankle member 750 includes an upper pin 766a extending through the tendon 752 and an upper portion of the insert 762 and a lower pin 766b extending through the tendon 752 and the lower attachment element 756 as shown in FIG. 19A. FIG. 19B illustrates an alternative embodiment lacking a lower pin. The tendon 752 therefore does not need a hole to accommodate a lower pin, which can advantageously improve the structural integrity of the tendon 752. In some embodiments, the upper pin 766a does not extend through the tendon and instead extends outwardly from the upper attachment element 754, for example, from a recessed portion 755 of the upper attachment element 754 that receives the upper portion of the insert 762. In such embodiments, the tendon 752 therefore does not need a hole to accommodate the upper pin, which can improve the structural integrity of the tendon 752. Ankle member 750 can include features similar to ankle members 550 and 650. For example, in the illustrated embodiment, ankle member 750 includes downwardly extending protrusions 760 at the front and/or back to advantageously guide or limit the range of motion of the upper attachment element 754 relative to the lower attachment element 756 in the forward and backward direction and protrusions or ridges 770a, 770b extending along a bottom surface of the lower attachment element 756 that are received in or engage corresponding grooves in a top surface of the arch portion of the foot member 610 or a member coupled to the top surface of the arch portion. Though the illustrated embodiment shows only two ridges 770a, 770b generally perpendicular to each other to engage two corresponding grooves in the foot member 610 or a disk coupled to the foot member 610, the system can have more than two ridges and corresponding grooves at various angles relative to each other. Furthermore, in some embodiments, the bottom surface of the lower attachment element 756 can include grooves instead of protrusions, and the top surface of the arch portion of the foot member 610 or a disk coupled to the top surface of the arch portion can include corresponding protrusions or ridges instead of grooves.

FIG. 21 illustrates an example embodiment of a tendon 552. As shown, the tendon 552 includes a central core 551 and a surrounding outer portion 553. The central core 551 is relatively stiff and prevents or inhibits compression, and the outer portion 553 is relatively more flexible (as compared to the central core 551). The central core 551 can be made of, for example, polyurethane 75D shore, and the outer portion 553 can be made of, for example, neoprene 80A shore. Other materials and/or material stiffnesses are also possible. The stiffer core and more flexible outer portion results in a type of pivot point in the middle of the tendon 552 such that the tendon 552 can rock forward or backward in the sagittal plane and/or side to side in the coronal plane rather than collapsing. This tendon design and the inserts 164 advantageously allow the user to more quickly engage the toe ground contacting portion 518 and heel ground contacting portion 520 when pressure is placed on the toe or heel (e.g., movement of the upper attachment element 554 relative to the lower attachment element 556 is stopped after a shorter or narrower range of motion in the forward-backward direction or in the sagittal plane, compared to a side-to-side direction, such that the overall foot 500 stiffens more quickly when weight or pressure is shifted toward the toe or heel). This advantageously improves a user's ability to control a board and maintain balance.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A prosthetic foot comprising:
an elongate foot member comprising a toe portion having a toe ground contacting portion, a heel portion having a heel ground contacting portion, and a substantially horizontal arch portion between the toe portion and heel portion;
wherein a width of the toe portion increases from the arch portion to the toe ground contacting portion and a width of the heel portion increases from the arch portion to the heel ground contacting portion.

2. The prosthetic foot of claim 1, wherein the toe portion and heel portion are symmetrical in a medial-lateral direction and the toe portion is symmetrical to the heel portion.

3. The prosthetic foot of claim 1, wherein the toe portion and heel portion have substantially the same length as measured from a center of the arch portion.

4. The prosthetic foot of claim 1, wherein the toe portion is longer than the heel portion.

5. The prosthetic foot of claim 1, wherein the toe ground contacting portion and heel ground contacting portion have substantially the same width.

6. The prosthetic foot of claim 5, wherein the toe portion and heel portion have substantially the same size and shape.

7. The prosthetic foot of claim 1, the toe and heel portions having openings therethrough.

8. The prosthetic foot of claim 1, further comprising an attachment member coupled to the arch portion and configured to couple the foot member to a user's residual limb or a prosthetic component.

9. The prosthetic foot of claim 1, wherein one or both of the toe portion and heel portion comprise medial and lateral blades configured to flex at least partially independently of each other.

10. A prosthetic foot comprising:
a foot member comprising a substantially horizontal arch portion, a toe portion extending downward and forward from the arch portion to a toe ground contacting portion at a toe end of the toe portion, and a heel portion extending downward and rearward from the arch portion to a heel ground contacting portion at a heel end of the heel portion, wherein a width of the toe portion increases from the arch portion to the toe ground contacting portion and a width of the heel portion increases from the arch portion to the heel ground contacting portion; and
an attachment member coupled to the arch portion and configured to couple the foot member to a user's residual limb or a prosthetic component, wherein the attachment member comprises:
a lower attachment element coupled to the arch portion;
an upper attachment element comprising a connector configured to couple to the user's residual limb or the prosthetic component; and
a resilient tendon extending between and coupled to the upper attachment element and the lower attachment element.

11. The prosthetic foot of claim 10, wherein the toe portion and heel portion are symmetrical.

12. The prosthetic foot of claim 10, wherein the toe portion and heel portion have substantially the same length as measured from a center of the arch portion.

13. The prosthetic foot of claim 10, wherein the toe portion is longer than the heel portion.

14. The prosthetic foot of claim 10, wherein the toe ground contacting portion and heel ground contacting portion have substantially the same width.

15. The prosthetic foot of claim 14, wherein toe portion and heel portion have substantially the same size and shape.

16. The prosthetic foot of claim 10, the toe and heel portions having openings therethrough.

17. The prosthetic foot of claim 10, wherein the attachment member is coupled to a center of the arch portion.

18. The prosthetic foot of claim 17, wherein the toe portion and heel portion are symmetrical in a medial-lateral direction and a fore-aft direction about an axis extending through the attachment member.

19. The prosthetic foot of claim 10, wherein the tendon is interchangeable.

20. The prosthetic foot of claim 10, wherein the upper attachment element comprises downwardly-extending ridges on opposite sides of the upper attachment element, and wherein the ridges are configured to control a range of motion of the upper attachment element.

21. The prosthetic foot of claim 20, wherein the upper attachment element is configured to be rotated to adjust positions of the ridges.

22. The prosthetic foot of claim 10, wherein one or both of the toe portion and heel portion comprise medial and lateral blades configured to flex at least partially independently of each other.
